(19) United States
(12) Patent Application Publication
(10) Pub. No.: US 2026/0294322 A1
(43) Pub. Date: Oct. 1, 2026

(54) SYSTEM AND METHOD FOR MODELING NEUROLOGICAL ACTIVITY

(71) Applicant: SURGICAL THEATER, INC., Beachwood, OH (US)

(72) Inventors: MORDECHAI AVISAR, HIGHLAND HEIGHTS, OH (US); ALON YAKOB GERI, ORANGE VILLAGE, OH (US); Gidon NAVROTSKY, Binyamina (IL)

(21) Appl. No.: 19/698,290

(22) Filed: Jun. 4, 2026

Related U.S. Application Data
(63) Continuation of application No. 17/427,652, filed on Feb. 12, 2020, now abandoned.

(51) Int. Cl. A61B 5/388; A61B 5/00; A61B 5/291; A61B 5/369; A61B 5/384; G16H 20/70; G16H 50/50

(52) U.S. Cl. CPC A61B 5/388; A61B 5/291; A61B 5/369; A61B 5/384; A61B 5/4094; G16H 20/70; G16H 50/50

(57) ABSTRACT

A system for modeling neurological activity includes a computer having one or more processors, one or more computer-readable tangible storage devices, and program instructions stored on at least one of the one or more storage devices. The program instructions are configured to receive electroencephalogram (“EEG”) data generated by an EEG device coupled to a plurality of electrodes disposed on a brain, the EEG data comprising a plurality of waveforms representative of electrical activity detected by the plurality of electrodes over a period of time; generate a graphical brain model representative of the brain; to convert the EEG data into a graphical EEG model representative of electrical activity; integrate the EEG model with the brain model, thereby enabling visualization of and interaction with the EEG model within the context of the brain model; and communicate the integrated EEG and brain model to a display.

### CROSS-REFERENCE TO RELATED APPLICATION

This application is a continuation of U.S. patent application Ser. No. 17/427,652 filed on Aug. 1, 2021, which is a national stage application of PCT application PCT/US20/17988 filed on Feb. 12, 2020, which claims priority from U.S. provisional patent application Ser. No. 62/804,432 filed on Feb. 12, 2019, all of which are incorporated by reference herein in their entirety

### FIELD OF DISCLOSURE

The present disclosure relates to the field of surgical procedures and more specifically to the field of epilepsy treatment.

### BACKGROUND

A seizure, characterized by a sudden surge of electrical activity in the brain, can be caused or triggered by a variety of factors, such a trauma, stroke, or an infection, for example. A seizure can affect a person's appearance or ability to function. Experiencing a series of seizures commonly results in a diagnosis of epilepsy, a chronic disorder characterized by unpredictable seizures. If left undiagnosed and untreated, epilepsy can cause health and social problems such as learning problems, sleeping problems, unexplained injuries, as well as risk of death.

Due to the complex nature of the disease, epilepsy management can be challenging. Administering medication is one possible method for treating epilepsy, although not always effective. For epileptic patients whose seizures cannot be managed by medication or diet, accurate identification and removal of the epileptogenic zone while minimizing new functional deficits from surgery is crucial. Performing a surgical procedure to remove an area of the brain that is causing the seizures is another and sometimes more effective treatment method. In order to perform such a surgical procedure, the area of the brain that is causing the seizures must first be identified.

Intercranial electroencephalographic (EEG) monitoring with subdural and/or depth electrodes is widely used for the surgical localization of the epileptogenic zone. In particular, a number of electrodes are placed at different points on the scalp and are connected by electrical wire to an EEG device. As the different electrodes detect electrical activity, the EEG device records the activity as a series of traces, each trace corresponding to a location in the brain. By analyzing the traces and identifying certain patterns of electrical activity, general locations in the brain may be identified as sources of a seizure. EEG anomalies are interpreted by trained neurologists, and electrode placement is performed by neurosurgeons using stereotactic guidance, or the stereoelectroencephalography (SEEG) method for implanting depth electrodes.

However, the ability to identify a location as a source of a seizure depends in part on the ability to effectively position the electrodes to enable accurate readings. Electrode placement planning can be a time-consuming process as it is done using standard DICOM dataset. Additionally, the technical complexity regarding SEEG electrode implantation leaves room for surgical errors and consequently poses high risks for complications, particularly for inexperienced surgeons. Misplacing the electrodes may result in inaccurate or incomplete data. Moreover, it may be difficult to interpret the series of traces and to effectively translate the traces into a visual location in the brain which a surgeon can use to efficiently and effectively remove the area causing the seizures. For example, because of inaccurate or difficult to interpret data, such a surgical procedure is commonly performed more than one time in order to completely remove the portion of the brain causing the seizure. Or in some cases, unnecessary portions of the brain may be removed. This may cause unnecessary trauma for the patient. While the SEEG method of depth electrode implantation has a long-reported successful record, there is still room for improvement with respect to optimal placement, seizure control, complication rates, and secondarily planning and surgical time. As a result, surgical intervention for treatment of seizures is commonly underutilized.

### SUMMARY

A system for modeling neurological activity includes a display and a computer. The computer includes one or more processors, one or more computer-readable tangible storage devices, and program instructions stored on at least one of the one or more storage devices for execution by at least one of the one or more processors. The program instructions are configured to receive electroencephalogram (“EEG”) data generated by an EEG device coupled to a plurality of electrodes disposed on a brain, the EEG data comprising a plurality of waveforms representative of electrical activity detected by the plurality of electrodes over a period of time; to generate a graphical brain model representative of the brain; to convert the EEG data into a graphical EEG model representative of electrical activity; to integrate the EEG model with the brain model, thereby enabling visualization of and interaction with the EEG model within the context of the brain model; and to communicate the integrated EEG and brain model to the display.

A method for modeling neurological activity incudes the steps of: receiving electroencephalogram (“EEG”) data generated by an EEG device coupled to a plurality of electrodes disposed on a brain, the EEG data comprising a plurality of waveforms representative of electrical activity detected by the plurality of electrodes over a period of time; generating a graphical brain model representative of the brain; converting the EEG data into a graphical EEG model representative of electrical activity; integrating the EEG model with the brain model, thereby enabling visualization of and interaction with the EEG model within the context of the brain model; and communicating the integrated EEG and brain model to a display.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example neurological activity modeling and treatment planning system.

FIG. 2 illustrates example EEG output.

FIG. 3 illustrates a block diagram of an example seizure modeling and treatment planning computer 300.

FIG. 4 illustrates an example model brain.

FIG. 5 illustrates an example model brain.

FIG. 6 illustrates an example model brain.

FIG. 7 illustrates an example view of a 3D model based on an example MD6DM model.

FIG. 8 illustrates an example view of a brain.

FIG. 9 illustrates an example view of a brain.

FIG. 10 illustrates an example view of a brain.

FIG. 11 illustrates an example view of a brain.

FIG. 12 illustrates an example graph for translating a set of data into a color-coded heat map.

FIG. 13 illustrates an example of a neurological activity modeling and treatment planning computer implemented in an enterprise solution.

FIG. 14 illustrates an example method for modeling neurological activity.

FIG. 15 illustrates an example computer system implementing the example neurological disorder computer of FIG. 1.

### DETAILED DESCRIPTION

The following acronyms and definitions will aid in understanding the detailed description:
AR—Augmented Reality—A live view of a physical, real-world environment whose elements have been enhanced by computer generated sensory elements such as sound, video, or graphics.
VR—Virtual Reality—A 3-Dimensional computer generated environment which can be explored and interacted with by a person in varying degrees.
HMD—Head Mounted Display refers to a headset which can be used in AR or VR environments. It may be wired or wireless. It may also include one or more add-ons such as headphones, microphone, HD camera, infrared camera, hand trackers, positional trackers etc.
Controller—A device which includes buttons and a direction controller. It may be wired or wireless. Examples of this device are Xbox gamepad, PlayStation gamepad, Oculus touch, etc.
SNAP Model—A SNAP case refers to a 3D texture or 3D objects created using one or more scans of a patient (CT, MR, fMR, DTI, etc.) in DICOM file format. It also includes different presets of segmentation for filtering specific ranges and coloring others in the 3D texture. It may also include 3D objects placed in the scene including 3D shapes to mark specific points or anatomy of interest, 3D Labels, 3D Measurement markers, 3D Arrows for guidance, and 3D surgical tools. Surgical tools and devices have been modeled for education and patient specific rehearsal, particularly for appropriately sizing aneurysm clips.
Avatar—An avatar represents a user inside the virtual environment.
MD6DM—Multi Dimension full spherical virtual reality, 6 Degrees of Freedom Model. It provides a graphical simulation environment which enables the physician to experience, plan, perform, and navigate the intervention in full spherical virtual reality environment.

A surgery rehearsal and preparation tool previously described in U.S. Pat. No. 8,311,791, incorporated in this application by reference, has been developed to convert static CT and MRI medical images into dynamic and interactive multi-dimensional full spherical virtual reality, six (6) degrees of freedom models (“MD6DM”) based on a prebuilt SNAP model that can be used by physicians to simulate medical procedures in real time. The MD6DM provides a graphical simulation environment which enables the physician to experience, plan, perform, and navigate the intervention in full spherical virtual reality environment. In particular, the MD6DM gives the surgeon the capability to navigate using a unique multidimensional model, built from traditional 2 dimensional patient medical scans, that gives spherical virtual reality 6 degrees of freedom (i.e. linear; x, y, z, and angular, yaw, pitch, roll) in the entire volumetric spherical virtual reality model.

The MD6DM is rendered in real time using a SNAP model built from the patient's own data set of medical images including CT, MRI, DTI etc., and is patient specific. A representative brain model, such as Atlas data, can be integrated to create a partially patient specific model if the surgeon so desires. The model gives a 360° spherical view from any point on the MD6DM. Using the MD6DM, the viewer is positioned virtually inside the anatomy and can look and observe both anatomical and pathological structures as if he were standing inside the patient's body. The viewer can look up, down, over the shoulders etc., and will see native structures in relation to each other, exactly as they are found in the patient. Spatial relationships between internal structures are preserved, and can be appreciated using the MD6DM.

The algorithm of the MD6DM takes the medical image information and builds it into a spherical model, a complete continuous real time model that can be viewed from any angle while “flying” inside the anatomical structure. In particular, after the CT, MRI, etc. takes a real organism and deconstructs it into hundreds of thin slices built from thousands of points, the MD 6DM reverts it to a 3D model by representing a 360° view of each of those points from both the inside and outside.

Described herein is a 360° AI system, leveraging a prebuilt SNAP model, the implements machine learning to first provide a “second opinion” and verify the neurologist's EEG interpretation and neurosurgeon's surgical plan and ultimately guide these decisions to provide safe and effective epilepsy treatment. The AI system described includes two subsystems: (1) a 360° AI Solution for Neurology; and (2) a 360° AI Solution for Neurosurgery. The 360° AI Solution for Neurology creates an Integrated 360° Anatomical Geo-Mapping/EEG Computer Vision Solution to show a 360° VR model rendered within the SNAP model with the inserted electrodes in the accurate spatial position within the brain. It shows the EEG graph outputs per electrode detection and helps the surgeon correlate between a physical position of an electrode to the graph readings detected by it. Additionally, the 360° AI solution uses specialized machine learning algorithms to accurately detect EEG anomalies and relate them to the specific 360° VR spherical position of the electrode itself, using heat maps to illustrate the epi center of the seizure. The 360° AI Solution for Neurosurgery calculates the safest entry points and trajectories for electrode implantation on a case-by-case basis. The 360° AI Solution learns to identify vessels within the patient-specific 360° VR (SNAP) model and find trajectories that steer clear of vessels to avoid intracranial bleeding. The 360° AI solution also finds multiple targets that may be detected by a single lead to minimize the number of necessary leads.

The 360° AI System for epilepsy surgery streamlines the epilepsy treatment process and improves workflow while providing accurate and precise electrode planning and EEG analysis, which can result in more successful localization of epileptogenic zones and consequently sustained seizure control or even seizure freedom. Specifically, the 360° AI System helps guide SEEG electrode placement by recommending the optimal electrode angle and entry point as well as the safest path to the target site without encountering vessels, sulci, and or other sensitive structures. By enabling efficient preoperative planning and safer intraoperative visualization and navigation, the system, leveraging a prebuilt patient specific SNAP model also shortens planning and surgical times of SEEG electrode implantation and reduce rates of complications such as intracranial bleeding and need for additional electrodes to aid in epileptogenic zone identification.

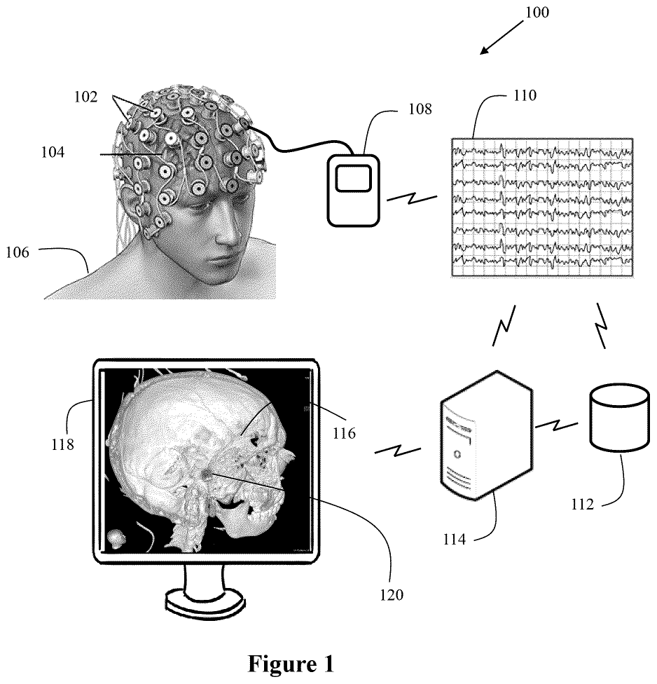

FIG. 1 illustrates an example neurological activity modeling and treatment planning system (“neurological activity system”) 100 that leverages a prebuilt SNAP model in order to enable visualization of a neurological disorder in 3D and to plan for treatment. In particular, the neurological disorder system 100 enables visualizing a 3D representation of a neurological disorder inside a 3D representation of a patient's brain as represented by the MD6DM model. It should be appreciated that although some examples described herein may refer specifically to modeling and visualizing a seizure in 3D, the example neurological disorder system 100 may similarly be used to visualize and model any neurological disorders such as Alzheimer's and sleep disorders, for example. It should be further appreciated that although the system 100 may be referred to herein with specific example to modeling and treating a neurological disorder, the system 100 may also be used to model any form of neurological activity.

The neurological disorder system 100 includes electrodes 102 for positioning on the skull 104 of a patient 106. The neurological disorder system 100 may include any suitable number of electrodes 102. The electrodes 102 detect electrical activity in the brain (not shown) under the skull 104. The electrodes 102 are coupled to an electroencephalography (“EEG”) device 108 which collects the electrical activity detected by the electrodes 102. The coupling between the electrodes 102 and the EEG device 108, as well as all other couplings referenced herein, may be either a wireless or a wired coupling.

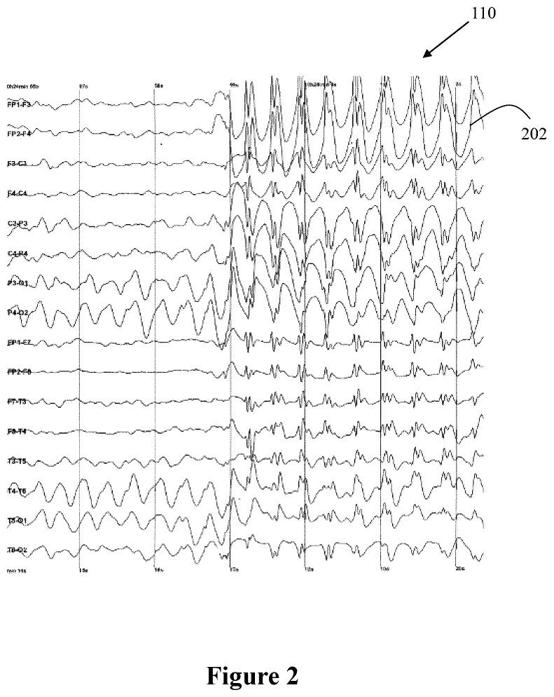

The EEG device 108 generates an EEG output 110 representative of the electrical activity. As illustrated in more detail in FIG. 2, the EEG output 110 includes a series of traces or waveforms 202 corresponding to the electrical activity detected by the different electrodes 102 at the different regions of the brain over a period of time. Referring back to FIG. 1, the neurological disorder system 100 further includes a database 112. In one example, the EEG device 108 stores the EEG output 110 in a database 112 for future retrieval and analysis. In one example, the database 112 further includes SNAP models representative of patient anatomies.

The neurological disorder system 100 further includes a neurological disorder modeling and treatment planning computer (“neurological disorder computer”) 114 that loads a prebuilt SNAP model. In one example, the neurological disorder computer 114 retrieves the prebuilt SNAP model 116 from the database 112. The neurological disorder system 100 renders and communicates a MD6DM model 116 based on a prebuilt SNAP model to a display 118. In one example, the display includes a head mounted display (“HMD”).

The neurological disorder computer 114 further receives the EEG output 110. In one example, the neurological disorder computer 114 retrieves previously generated EEG output 110 from the database 112. In another example, the neurological disorder computer 114 receives the EEG output 110 in real time directly from the EEG device 108.

The neurological disorder computer 114 generates a 3D neurological disorder model 120 based on the EEG output 110. In particular, the neurological disorder computer 114 converts data represented by the traces 202 into a 3D neurological disorder model 120. In one example, the neurological disorder computer 114 generates a heat map representative of the strength or presence of the neurological disorder (such as a seizure), based on electrical activity, at different positions within the brain.

The neurological disorder computer 114 modifies the MD6DM model 116 and incorporates the 3D neurological disorder model 120 into the MD 6DM model 116 as communicated to the display 118 such that a user can visualize and interact with the 3D neurological disorder model 120 within the context of the MD6DM model 116. In other words, the user is able to virtually see and interact with the neurological disorder inside the brain. This enables the user, such as a physician, to effectively and efficiently interpret the EEG data 110 and to accurately identify a location in the brain as a source of the neurological disorder. Thus, in the case of a seizure for example, the physician can effectively plan for surgery and remove the area from the brain causing the seizure while reducing chances of error as well as reducing possibly of unnecessary trauma for the patient.

In one example, the neurological disorder computer 114 aids in providing recommendations for positioning of the electrodes 102 on the skull 104 of the patient 106. This enables more accurate and placement, thereby eliminating errors and increasing accuracy of resulting collected data. In one example, the neurological disorder computer 114 uses artificial intelligence algorithms to train or learn, using historical data of previous electrode placements, and to make recommendations based on the training.

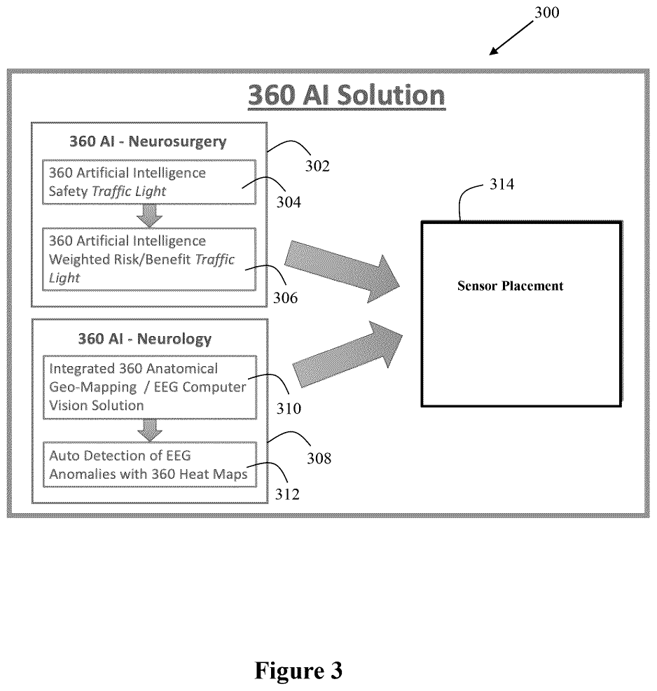

The neurological disorder computer 114 will be further appreciated with specific reference to an example seizure modeling and treatment application of the neurological disorder system 100. FIG. 3 illustrates a block diagram of an example seizure modeling and treatment planning computer 300 (i.e. the neurological disorder computer 114 of FIG. 1), also referred to herein as the AI Solution or System. The AI System 300 includes three modules: an AI Neurosurgery Module 302; an AI Neurology Module 308; and a Sensor Placement Module 314, which will be described in further detail herein.

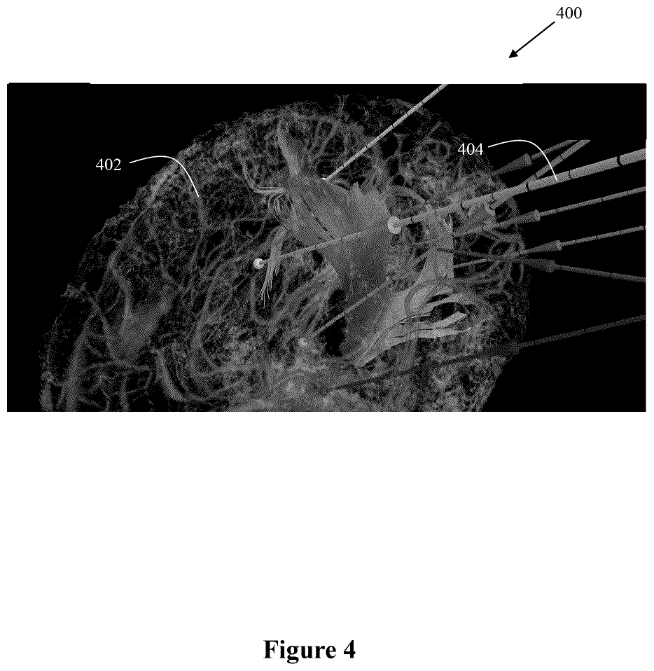

The AI Neurosurgery Module 302 provides guidance for entry into a patient's brain for placing electrodes for modeling and treating epilepsy and seizures. In particular, the AI Neurosurgery Module 302 includes an artificial intelligence safety traffic light sub-module 304 for suggesting to a surgeon the safest approaches for the entry points and trajectories of the electrodes including the sensors. For example, in the model brain 400 illustrated in FIG. 4, the artificial intelligence safety traffic light sub-module 304 learns to identify vessels 402 in the brain 400 and to find trajectories for lead 404 placement safely that go as far away from the vessels 402 as possible.

Referring again to FIG. 3, in one example, 360 artificial intelligence safety traffic light sub-module 304 maps the surface of a skull according to colors to differentiate proximity to vessels. For example, the 360 artificial intelligence safety traffic light sub-module 304 may map a skull with red, yellow, and green colors. In such an example, red may indicate that a trajectory with the closest proximity to a vascular structure (i.e. less than 1 mm), yellow may indicate that a trajectory through this point is close to a vessel (i.e. less than 2 mm), and green may indicate that a trajectory through this point is reasonably far from a vessel (i.e. more than 2 mm).

The AI Neurosurgery Module 302 further includes an artificial intelligence weighted risk/benefit traffic light sub-module 306. The artificial intelligence weighted risk/benefit traffic light sub-module 306 minimizes the number of necessary leads by finding several targets that may be detected by a single lead.

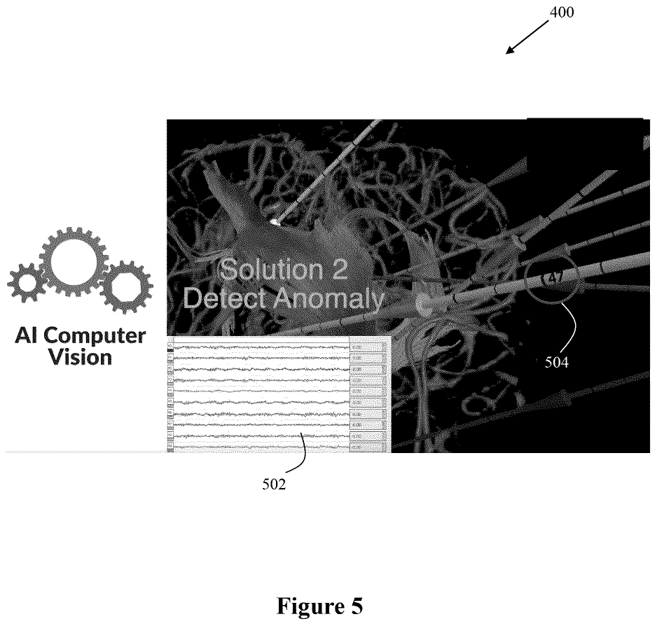

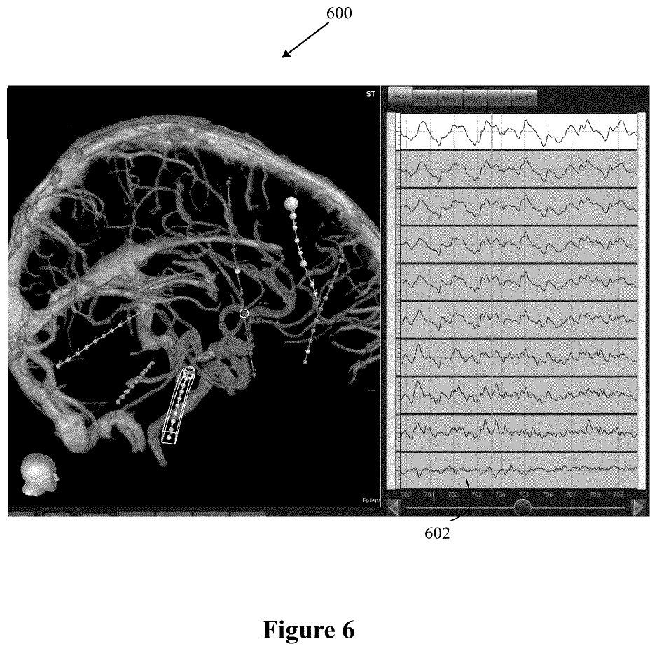

The AI Neurology Module 308 includes an integrated Anatomical Geo-mapping/EEG Computer vision module 310 who's objective is to enable answering the question where the anomaly in the EEG is located in the brain. The integrated Anatomical Geo-mapping/EEG Computer vision module 310 creates an integrative solution that incorporates the several steps in the epilepsy treatment continuum. In particular, the integrated Anatomical Geo-mapping/EEG Computer vision module 310 shows a 360 model with the inserted contacts in the accurate spatial position within the brain, as illustrated in FIG. 4. FIG. 5 further illustrates the model brain 400, including EEG graph outputs per sensor detection 502 (i.e. the EEG output 110 of FIG. 2) and helps the surgeon correlate between a physical position of a sensor to the graph readings detected by it. The integrated Anatomical Geo-mapping/EEG Computer vision module 310 uses CT/MRI scans of an anatomy, including the sensors, and detects automatically the sensor positions within the 360-anatomy re-creation (i.e. the prebuilt SNAP model). Based on this information, Anatomical Geo-mapping/EEG Computer vision module 310 is able to identify seizure coordinates 504 within the brain 400. FIG. 6 illustrates another example model brain 600, including correlation with the EEG graph outputs per sensor detection 602 (i.e. the EEG output 110 of FIG. 2).

Referring again to FIG. 3, the AI Neurology Module 308 further includes an auto detection of EEG anomalies with 360 heat maps module 312. The auto detection of EEG anomalies with 360 heat maps module 312 learns to detect the anomalies in the graph readings automatically. It uses specialized machine learning algorithms to accurately detect those anomalies and relate them to the specific 360 spherical position of the sensor itself, using heat maps to illustrate the epi center of the seizure.

The Sensor Placement Module 314 suggests to the surgeon the interest points in the brain where he should locate the sensors. The Sensor Placement Module 314 takes into account many variables, such as past EEGs, patient age, and patient health history, etc.

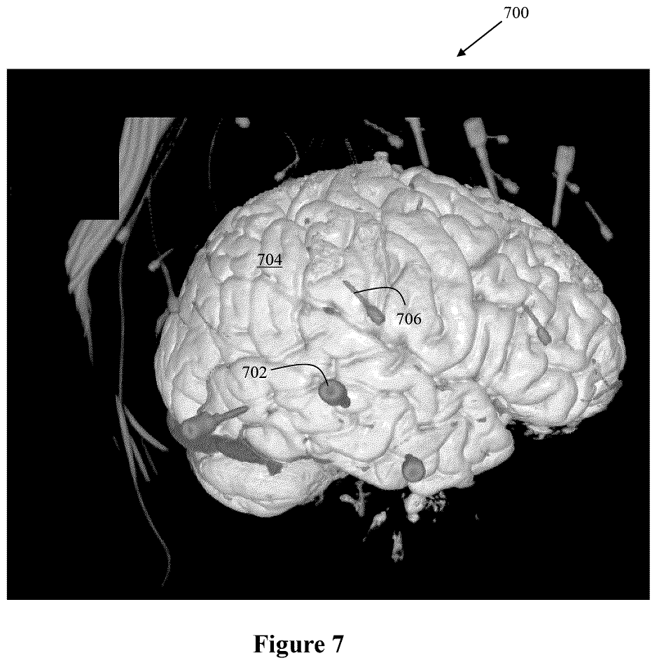

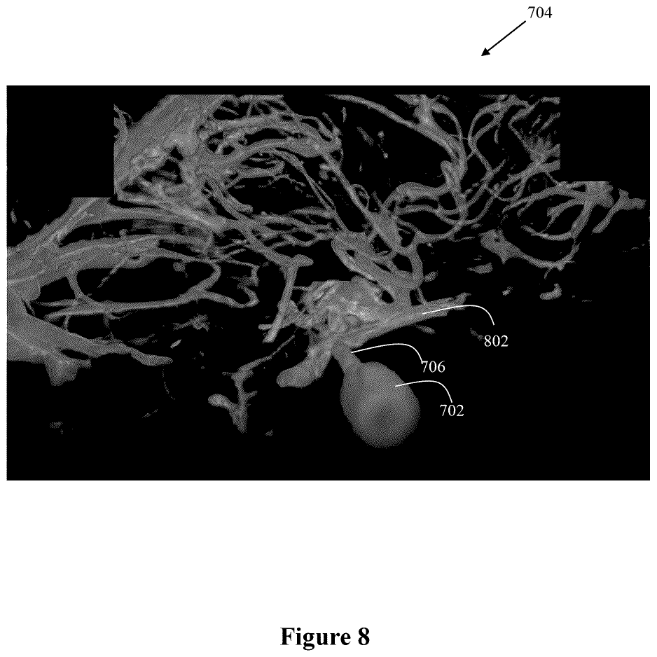

Using a heat map to illustrate the epi center of the seizure will be further appreciated with reference to FIGS. 7-12. FIG. 7 illustrates an example view of a 3D model 700 based on an example MD6DM model. The 3D model 700 includes blue electrodes 702 (i.e. electrodes 102 of FIG. 1) disposed on a brain 704. In one example, stereo-EEGs may be extracted from a CT scan and artifacts may be segmented out using AI so that leads 706 connected to electrodes 702 become visible. FIG. 8 illustrates a closeup of the brain 804, showing the electrodes 702 connected to leads 706 which are navigating between various vessels 802.

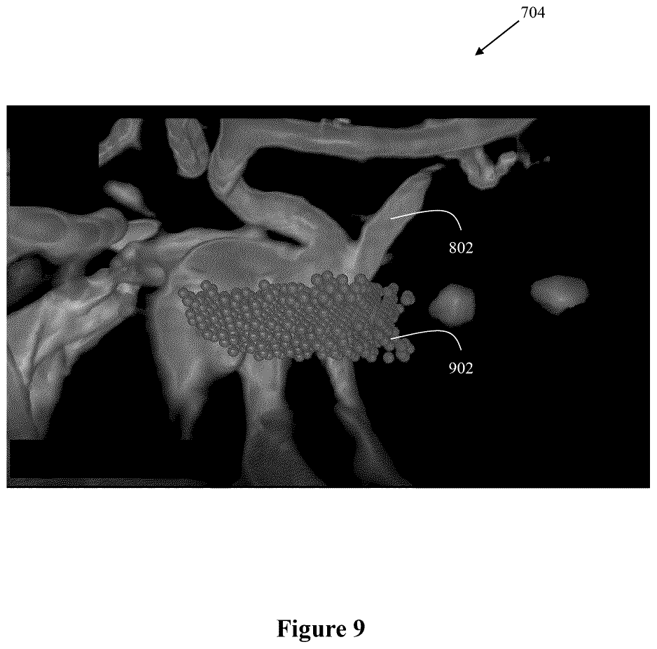

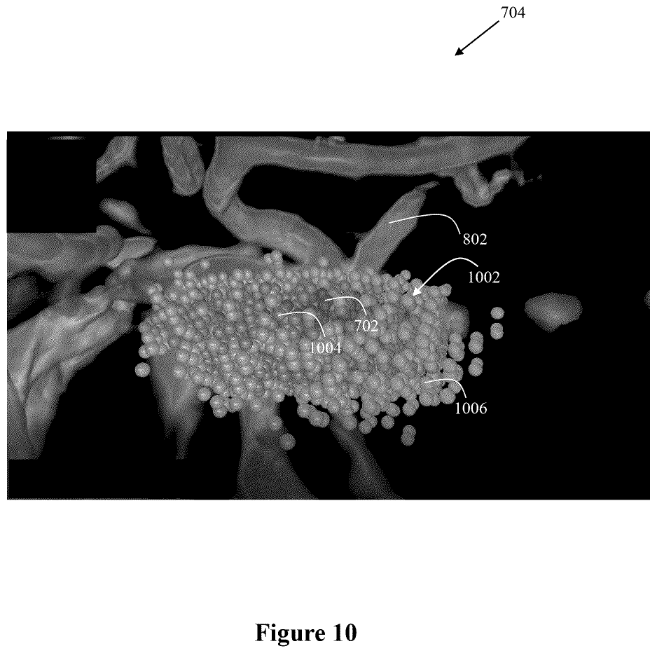

The focus 902 of a seizure is visually depicted within and among the vessels 802, as illustrated in FIG. 9. In one example, the focus of the seizer is depicted with red dots. This indicates the most likely location of occurrence of the seizure based on the data collected and analyzed. FIG. 10 illustrates a model of seizure 1002 between various vessels 802. The seizure model 1002 is based on the focus 1002, and also includes middle regions 1004 and outer regions 1006. Middle regions 1004 are representative of regions that are less likely to be the center of seizure activity within the seizure model 1002 but are still likely to be a source of some of the seizure activity. The middle region 1004 may be represented in an organ color, in one example. The outer region 1006 are representative of regions that are the end areas of the seizure region of the seizure model 1002 but still possibly a source of some seizure activity. The outer region 1006 may be represented in a yellow color, for example. Thus, the three regions (i.e. the focus 902, the middle 1004, and the outer 1006, combine to form a seizure model 1002 based on a heat map indicative of the likelihood of presence of seizure activity within the brain 704. Although the example seizure model 1002 is depicted to include three layers or regions represented by three different colors, a seizure model 1002 may similarly include any suitable number of regions represented by any suitable combination of colors.

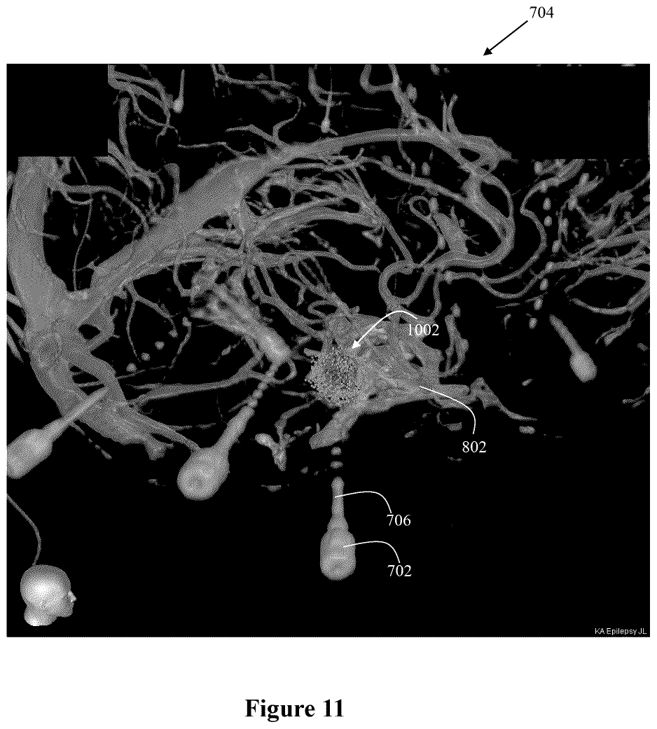

FIG. 11 illustrates another view of the brain 704, in which the seizure model 1002 is depicted at the end of the electrodes 702 connected to leads 706 which are navigating between various vessels 802. Although the example illustrates the seizure model 1002 being associated with a single electrode 702 and lead 706, a seizure model 1002 may be identified by and associated with any suitable number of electrodes 702 and leads 706.

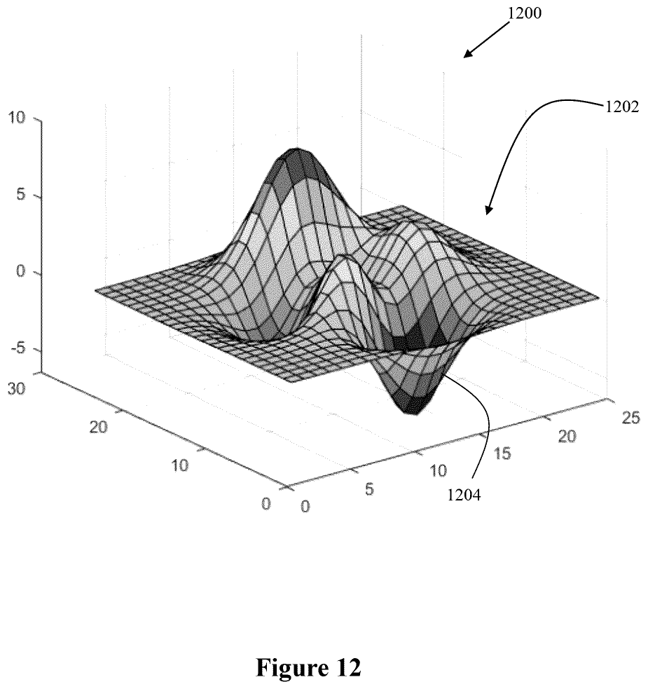

FIG. 12 illustrates an example graph 1200 used to translate a set of data (not shown) into a color-coded heat map 1202 indicative of the strength or presence of data points within certain regions 1204. Such a graphing technique may be applied to collected EEG data in order to generate a heat map representative of a seizure model (i.e. the seizure model 1002 of FIGS. 10-12).

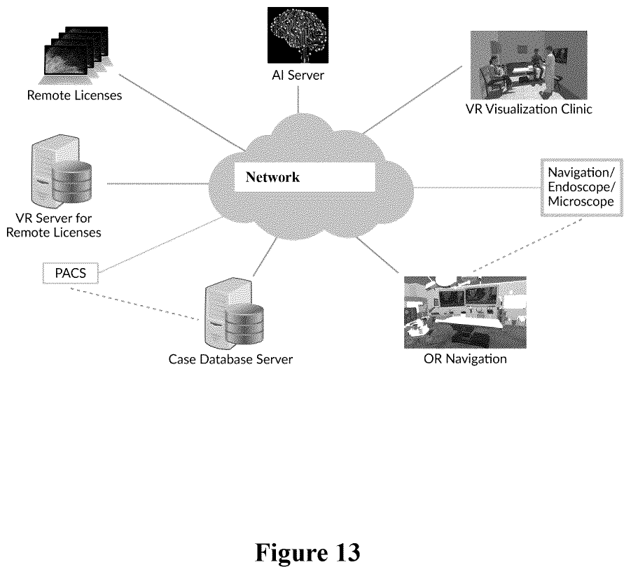

In on example, as illustrated in FIG. 13, a neurological disorder modeling and treatment planning computer (i.e. the seizure modeling and treatment planning computer 300 of FIG. 3 or the neurological disorder computer 114 of FIG. 1) may be deployed in an enterprise model/solution.

Integrated with an AI Server, a neurological disorder modeling and treatment planning computer connects to the hospitals network while complying with its security policies. All 360° VR cases (prebuilt SNAP cases) are stored in the hospital's data center and are accessible to any authorized Application on the network, such as the neurological disorder modeling and treatment planning computer. The Applications can either be run on a dedicated machines, or can be run on a remote client with reduced capabilities.

The AI Server monitors and collect data in a secured environment to feed the machine learning and deep learning algorithms, which will be enhanced with every additional 360° dataset. The AI Server runs all the Artificial Intelligence algorithms required for the Epilepsy cases. In particular, the AI Server runs two types of algorithms. First, the AI Server runs Learning Algorithms. In particular, the AI Server connects to the hospital networks (i.e. PACS, EHR) and feeds on the previous epilepsy cases that are stored on them. It then updates its deep neural networks accordingly. Second, the AI Server runs Suggestion Algorithms. The deep neural networks will help the physicians with suggestions of approaches to dealing with new Epilepsy cases, including 360° Leads placement and Anomaly detection.

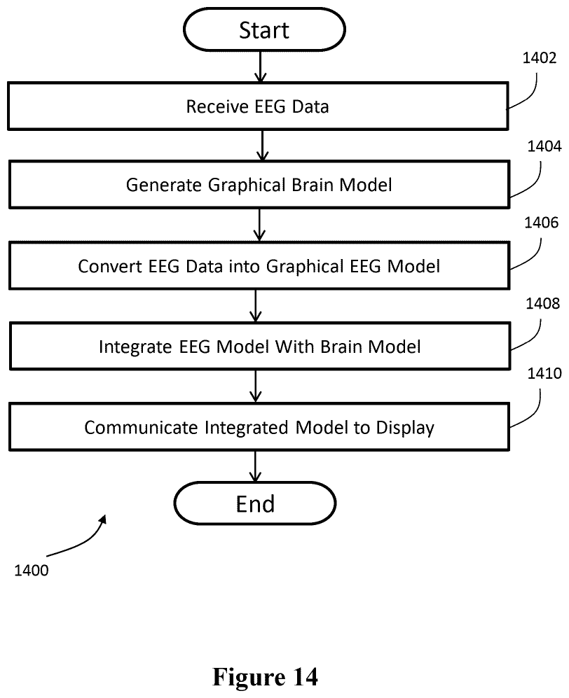

FIG. 14 illustrates an example method for modeling neurological activity. At 1402, the neurological modeling computer 114 receives electroencephalogram (“EEG”) data generated by an EEG device coupled to a plurality of electrodes disposed on a brain. The EEG data includes a waveforms representative of electrical activity detected by the electrodes over a period of time. At 1404, the neurological modeling computer 114 generates a graphical brain model representative of the brain. At 1406, the neurological modeling computer 114 converts the EEG data into a graphical EEG model representative of electrical activity. At 1408, the neurological modeling computer 114 integrates the EEG model with the brain model, thereby enabling visualization of and interaction with the EEG model within the context of the brain model. At 1408, the neurological modeling computer 114 communicates the integrated EEG and brain model to the display 118.

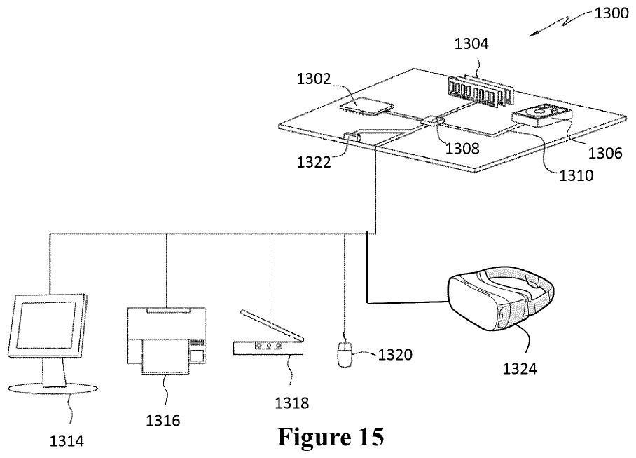

FIG. 15 is a schematic diagram of an example computer for implementing the example neurological modeling computer 114 of FIG. 1. The example computer 1500 is intended to represent various forms of digital computers, including laptops, desktops, handheld computers, tablet computers, smartphones, servers, and other similar types of computing devices. Computer 1500 includes a processor 1502, memory 1504, a storage device 1506, and a communication port 1508, operably connected by an interface 1510 via a bus 1512.

Processor 1502 processes instructions, via memory 1504, for execution within computer 1500. In an example embodiment, multiple processors along with multiple memories may be used.

Memory 1504 may be volatile memory or non-volatile memory. Memory 1504 may be a computer-readable medium, such as a magnetic disk or optical disk. Storage device 1506 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 1504 or storage device 1506.

Computer 1500 can be coupled to one or more input and output devices such as a display 1514, a printer 1516, a scanner 1518, a mouse 1520, and a HMD 1524.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, C #, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

1. A method of preparing a surgical plan using a 3D model of the anatomy of a patient using a computer modeling system including a computer, said method comprising:
placing a plurality of electrodes on or near a biological feature of the anatomy of the patient;
determining electrode locations of the plurality of electrodes with respect to the biological feature;
obtaining biological images of the biological feature the patient;
sending electrode data from said electrodes about a functioning of the biological feature of the patient to the computer modeling system;
preparing a dynamic model of the biological feature, comprising the computer modeling system being configured to perform a method comprising:
generating a 3D model of the biological feature of the patient using said biological images;
generating images representing the functioning of the biological feature of the patient from the electrode data;
generating an integrated dynamic model including dynamic model images representing said 3D model of the biological feature of the patient showing placement locations of said electrodes with respect to said biological feature as provided by said determined electrode locations, said dynamic model images including said images representing the functioning of the biological feature, and
displaying said dynamic model images on a display; and
preparing a surgical plan using said integrated dynamic model.

2. The method of claim 1 wherein the biological feature is the brain of the patient, and wherein said electrodes are EEG electrodes.

3. The method of claim 1, wherein the biological feature is the brain of the patient, and wherein the electrode data includes data generated from detecting a brain seizure.

4. The method of claim 3, wherein the computer modeling system is further configured to convert the electrode data into a graphical seizure model representative of electrical activity in the brain by generating a heat map representative of the strength of the neurological activity at different positions within the brain.

5. The method of claim 3, wherein the computer modeling system is further configured to generate a plurality of activity waveforms from the electrode data for display to a user.

6. The method of claim 5, wherein the computer modeling system is further configured to analyze said plurality of waveforms to detect anomalies in the waveforms and to correlate the anomalies to positions of the electrodes.

7. The method of claim 1, wherein the biological feature is the brain of the patient, and wherein said electrodes are EEG electrodes, and wherein the computer modeling system is further configured to receive the electrode data in real time directly from an EEG device connected to said EEG electrodes such that said dynamic model images show said functioning of the brain in real time.

8. The method of claim 1, wherein the biological feature is the brain of the patient and the electrodes are EEG electrodes, and wherein the computer modeling system is further configured to provide a recommendation for safely placing the EEG electrodes on a skull of the patient by analyzing the 3D model to determine locations of blood vessels and determining at least one location on the skull for placing at least one electrode such that the proximity between the blood vessels and a trajectory stemming from the location of the at least one electrode to be placed is minimized.

9. The method of claim 8, wherein the computer modeling system is further configured to map a surface of the brain such that the map is indicative of a proximity between the vessels and a trajectory stemming from a location on the map.

10. The method of claim 8, wherein the computer modeling system comprises an executing artificial intelligence application configured to learn from historical epilepsy data of the patient and to provide suggestions for future epilepsy treatment, including recommending placement location of at least one of electrode and including detecting anomalies.

11. A method of preparing a surgical plan using a 3D model of the brain of a patient using a computer modeling system including a computer, said method comprising:
placing a plurality of EEG electrodes on or near the brain of the patient;
determining EEG electrode locations of the plurality of EEG electrodes with respect to the brain;
obtaining biological images of the brain the patient;
sending EEG electrode data from said EEG electrodes about electrical activity of the brain to the computer modeling system;
preparing a dynamic model of the brain, comprising the computer modeling system being configured to perform a method comprising:
generating a 3D model of the brain using said biological images;
generating activity images representing the electrical activity of the brain of the patient from the EEG electrode data;
generating an integrated dynamic model including dynamic model images representing said 3D model of the brain showing placement locations of said EEG electrodes with respect to said brain as provided by said determined EEG electrode locations, said dynamic model images including said activity images representing the electrical activity of the brain, and
displaying said dynamic model images on a display; and
preparing a surgical plan for the brain of the patient using said integrated dynamic model.

12. The method of claim 11 wherein the 3D model of the brain and the integrated dynamic model comprise a three-dimensional model.

13. The method of claim 11, wherein the electrical activity represented by the EEG data comprises a brain seizure, and wherein said activity images are configured to show regions where seizure origination is most likely.

14. The method of claim 11, wherein said activity images include a heat map representative of the strength of neurological activity at different positions within the brain.

15. The method of claim 11, further comprising generating a plurality of waveforms representing the electrical activity of the brain; and correlating positions of the EEG electrodes with the plurality of waveforms.

16. The method of claim 15, further comprising detecting anomalies in the waveforms and correlating the anomalies to positions of the EEG electrodes.

17. The method of claim 11, wherein the computer modeling system is further configured to provide a recommendation for safely placing the EEG electrodes on a skull of the patient by analyzing the 3D model of the brain to determine locations of blood vessels and determining at least one location on the skull for placing at least one electrode such that the proximity between the blood vessels and a trajectory stemming from the location of the at least one electrode to be placed is minimized.

18. The method of claim 11, wherein the computer modeling system comprises an executing artificial intelligence application configured to learn from historical epilepsy data of the patient and to provide suggestions for future epilepsy treatment, including recommending placement location of at least one of the EEG electrodes and including detecting anomalies.

19. The method of claim 11, wherein the computer modeling system is further configured to receive the EEG electrode data in real time directly from an EEG device connected to said EEG electrodes such that said dynamic model images show said functioning of the brain in real time.

20. A method of providing a dynamic 3D model of the brain of a patient for preparing a surgical plan using a computer modeling system including a computer, said method comprising:
placing a plurality of EEG electrodes on or near the brain of the patient;
determining EEG electrode locations of the plurality of EEG electrodes with respect to the brain;
obtaining biological images of the brain the patient;
generating EEG electrode data about electrical activity of the brain from the EEG electrodes in real time;
sending the EEG electrode data to the computer modeling system;
preparing the dynamic3D model of the brain, comprising the computer modeling system being configured to perform a method comprising:
generating a 3D model of the brain using said biological images;
generating activity images representing the electrical activity of the brain of the patient from the EEG electrode data;
generating an integrated dynamic model including images representing said 3D model of the brain showing placement locations of said EEG electrodes with respect to said brain as provided by said determined EEG electrode locations, said dynamic model images including said activity images representing the electrical activity of the brain, and
displaying said dynamic model images on a display.
